(19) United States
(12) Patent Application Publication
(10) Pub. No.: US 2009/0324444 A1
(43) Pub. Date: Dec. 31, 2009

(54) DOOR OPENING SYSTEM

(76) Inventor: Greg Stratmann, San Francisco, CA (US)

Correspondence Address: CLARK & ELBING LLP, 101 FEDERAL STREET, BOSTON, MA, 02110, US

(21) Appl. No.: 12/114,519

(22) Filed: May 2, 2008

Related U.S. Application Data
(60) Provisional application No. 61/047,281, filed on Apr. 23, 2008.

(51) Int. Cl. A61L 2/18; E05F 15/00

(52) U.S. Cl. 422/28; 49/324

(57) ABSTRACT

The invention relates generally to the field of hand sanitation, and particularly to a door opening system that restricts the movement of people through a doorway or threshold to those that have activated a hand sanitizer dispensing unit.

### CROSS-REFERENCE TO RELATED APPLICATIONS

This application claims priority from provisional patent application U.S. Ser. No. 61/047,281, filed Apr. 23, 2008, hereby incorporated by reference.

### FIELD OF THE INVENTION

The invention relates generally to the field of hand sanitation, and particularly to a door opening system that restricts the movement of people (e.g., physicians, nurses, and patients) through a doorway or threshold to those that have activated a hand sanitizer dispensing unit. The invention thus compels the user to receive an effective amount of a hand sanitizer in order to pass the threshold. The selective admission of people who have used a hand sanitizer can reduce the incidence of microbial infections.

### BACKGROUND OF THE INVENTION

Healthcare-associated infections are a common cause of morbidity and mortality in the United States and are among the most common adverse events in healthcare. Agents or pathogens that can be acquired or transmitted in a healthcare setting include: Acinetobacter baumannii; Burkholderia cepacia; chickenpox (varicella); Clostridium difficile; Clostridium sordellii; Creutzfeldt-Jakob Disease (CJD); ebola virus (viral Hemorrhagic Fever); hepatitis viruses A, B, and C; HIV/AIDS; influenzaviruses; MRSA (methicillin-resistant Staphylococcus aureus); mumps; norovirus; streptococcal species, Pseudomonas Aeruginosa, parvovirus; poliovirus; pneumonia; rubella; SARS; S. pneumoniae; tuberculosis; VISA (vancomycin intermediate Staphylococcus aureus); and VRE (vancomycin-resistant enterococci).

### SUMMARY OF THE INVENTION

In a first aspect, the invention provides a door opening system that has a hand sanitizer dispensing unit that releases a hand sanitizer upon activation, a door with a door opening or locking device, and an electrical, mechanical, or wireless signal connection between the hand sanitizer dispensing unit and the opening or locking device, wherein the door opening or locking device is adapted to be activated to allow movement of the door upon activation of the hand sanitizer dispensing unit.

In one embodiment of the invention, the hand sanitizer is a chemical or radiological sanitizer. Chemical sanitizers include triclosan, ethanol, isopropanol, n-propanol, povidone-iodide, chlorhexidine gluconate, octenidine, chloroxylenol, hexachlorophene, alkyl benzalkonium chloride, benzethonium chloride, cetrimide, and cetylpyridium chloride. In preferred embodiments, the chemical sanitizer is ethanol or triclosan. The chemical hand sanitizer can be applied with a towel, towelette, or hand wipe. The radiological sanitizer can be ultraviolet light.

In another embodiment of the invention, the door is an elevator door. The wireless signal connection can be a radio or infrared connection. The door can have a manual override mechanism that allows movement of the door independent of activation of the hand sanitizer dispensing unit. The hand sanitizer dispensing unit can be mounted on the door. The system can have instructions for use.

In a further embodiment of the invention, the door is in a hospital, medical facility, nursing home, school, daycare center, library, museum, public meeting or working space, retail store, grocery store, restaurant, cafeteria, kitchen, food production or processing facility, public transportation station, or public transit vehicle. In one embodiment, the door is in a hospital. In another embodiment, the door is in a hospital emergency room (ER), intensive care unit (ICU), operating room (OR), surgical preparation room, outpatient facility, birthing room, pharmacy, cafeteria, restroom, elevator bank, or waiting room. In one embodiment, the door is in an airport, train station, subway station, or bus station. In another embodiment door is in an airplane, a train, a subway train, a bus, or boat. The boat can be a cruise ship.

In another embodiment of the invention, the door separates medical caregivers from medical patients. Medical caregivers include physicians, nurses, medical technicians, pharmacists, and medical students.

In a second aspect, the invention provides a method of reducing microbial infection by activating a hand sanitizer dispensing unit mounted on or proximal to a door with a door opening or locking device, wherein activation allows movement of the door.

### Definitions

By “door” is meant a moveable barrier meant to separate two distinct spaces within a structure. The door can be a single, double, sliding, or revolving door. The door can be opened manually or automatically by a door opening device, as defined herein. Elevator doors and other doors found in the interior or exterior of modes of transportation are also suitable doors for use in the present invention. The door can be located in a stationary structure (e.g., a hospital, airport) or in a moving transport object (e.g., a train, boat, or airplane). The door can separate two public or private spaces or separate a public space from a private space (e.g., a door between a hospital hallway and a patient room).

By “effective amount” is meant the measured quantity of a chemical or radiological hand sanitizer that is necessary to reduce (e.g., by 5%, 10%, 20%, 30%, 40%, 50%, 60%, 70%, 80%, 90%, 95%, or 99%) or eliminate infectious microbes from the skin surface of the hands.

By “hand sanitizer” or “sanitizer” is meant any compound or means used to remove, reduce, or kill microbes and pathogens (e.g., bacteria and viruses) upon application to the hands or lower arms of a human (e.g., a healthcare professional) that does not require removal following use, as required with standard hand-washing with soap and water. Hand sanitizers can be chemically or radiologically-based. A chemical sanitizer can be in a liquid, gel, foam, or powder form. Alternatively, the sanitizer can be present on or within a towel (e.g., a towelette) or napkin. Active ingredients in a chemical hand sanitizer include but are not limited to: triclosan, ethanol (e.g., 62% w/w ethanol, such as Purell®), isopropanol, n-propanol, povidone-iodide, chlorhexidine gluconate, octenidine, chloroxylenol, hexachlorophene, alkyl benzalkonium chloride, benzethonium chloride, cetrimide, and cetylpyridium chloride. Chemical hand sanitizers are discussed in further detail in the Center for Disease Control and Prevention's Morbidity and Mortality Weekly Report entitled “Guideline for Hand Hygiene in Health-Care Settings” (Vol. 51, Oct. 25, 2002). A radiological-based hand sanitizer is a device that, upon activation, exposes a user's hands or lower arms to radiation (e.g., ultraviolet light) that is germicidal but not harmful to the user. See, e.g., U.S. Pat. No. 6,254,625, incorporated herein by reference.

### BRIEF DESCRIPTION OF THE DRAWINGS

FIG. 1 is a perspective view of the door opening system of the invention in the context of a single door threshold.

FIG. 2 is a perspective view of the door opening system of the invention in the context of a double door threshold.

### DETAILED DESCRIPTION OF THE INVENTION

In the following description, for purposes of explanation and not limitation, specific details are set forth in order to provide a thorough understanding of the present invention. However, it will be apparent to one skilled in the art that the present invention may be practiced in other embodiments that depart from these specific details. In other instances, detailed descriptions of well-known methods and devices are omitted so as to not obscure the description of the present invention with unnecessary detail.

### The Door Opening System

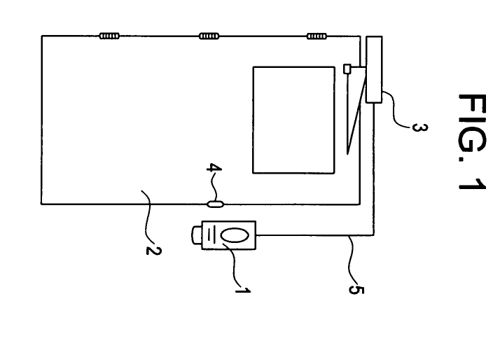

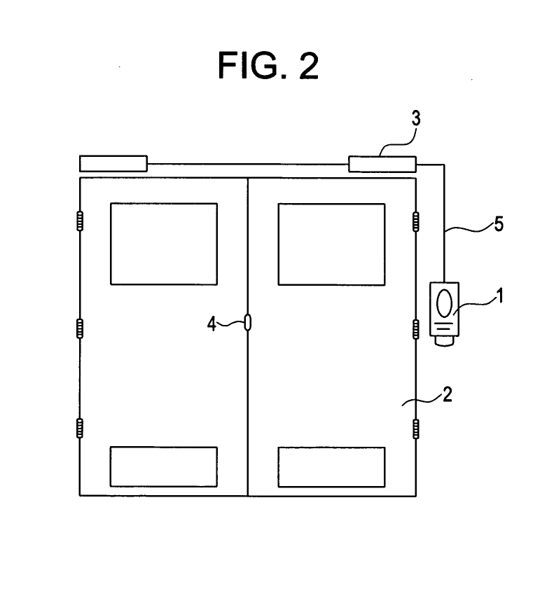

Exemplary door opening systems constructed in accordance with the present invention are shown in FIGS. 1 and 2. The door opening system includes a hand sanitizer dispensing unit, a door with a opening or locking device, and an electrical, mechanical, or wireless signal connection between the dispensing unit and the opening or locking device. Any person (i.e., a “user”) desiring entry into a space blocked by a closed or locked door is compelled to activate the hand sanitizer dispensing unit. Activation of the dispensing unit initiates two events: 1) the unit dispensation of an effective amount of a chemical or radiological hand sanitizer on to one or both hands of the user and 2) transmission of a signal from the dispensing unit that unlocks or opens the door to allow the user entry past the threshold. Each component of the door opening system is described in further detail below, as numbered in FIGS. 1 and 2.

### 1. Hand Sanitizer Dispensing Unit

A hand sanitizer dispensing unit is a device that, upon activation, dispenses, applies, or exposes a predetermined and effective amount of a hand sanitizer to the hands of a user. The hand sanitizer can be chemical (e.g., an alcohol-based sanitizer) or radiological (e.g., ultraviolet light), as defined herein.

A chemical hand sanitizer dispensing unit dispenses a measured quantity (e.g., 0.5 mL, 0.75 mL, 1.0 mL, 1.25 mL, 1.5 mL, 1.75 mL, 2.0 mL, 2.5 mL, 3.0 mL, 3.5 mL, 4.0 mL, 4.5 mL, 5.0 mL or more) of a chemical hand sanitizer that, when applied evenly over the surface of both hands, is effective in reducing or eliminating microbes. A chemical hand sanitizer dispensing unit consists of a structural or ornamental casing, a reservoir to hold a quantity of chemical hand sanitizer, a dispensing head or nozzle, and an activation trigger, such as a button, lever, or pull arm. Upon dispensation of a chemical hand sanitizer, the user rubs his or her hands together to evenly apply the sanitizer.

A hand sanitizer dispensing unit can also dispense a towel (e.g., a towelette or disposable hand wipe; see e.g., U.S. Pat. No. 4,620,502) containing or infused with a chemical hand sanitizer. The towel can be dispensed ready-to-use, or can be packaged (e.g., in foil wrapping) to preserve moisture content. Upon receipt of the towel, the user contacts it to his or her hands to apply the chemical hand sanitizer evenly over the surface of the skin.

A radiological hand sanitizer unit emits radiological energy (e.g., ultraviolet light) upon activation. Exposure of the users hands to this energy is effective in reducing or eliminating infectious microbes from the surface of the skin.

A hand sanitizer dispensing unit must be activated in order to release a hand sanitizer or sanitizing towel. A hand sanitizer dispensing unit is activated by the user by providing an affirmative stimulus to the unit. Stimuli include, e.g., pushing or pulling a lever or stepping upon or touching a pressure-sensitive pad or button. Alternatively, a dispensing unit can be activated by sound (e.g., a voice or hand clap), proximity (e.g., optical (i.e., visible spectrum, magnet strips, ID cards), infrared, or thermal detection of user proximity), or motion (e.g., detection of a hand or finger close (e.g., 6 inches or less) to the dispensing unit).

A hand sanitizer dispensing unit can be mounted to a wall or door. Alternatively, a dispensing unit can be free-standing (e.g., placed on a desk or table), suspended (e.g., from the ceiling), or supported by an integrated or attached stand.

The location of the hand sanitizer dispensing unit is important for efficient practice of the methods of the invention. Ideally, the dispensing unit is located on or near the door to which it regulates passage. Instructional signs can be placed near the door or dispensing unit to advise a person unfamiliar with the door opening system of the invention (i.e., activating the dispensing unit to open the door) how to activate the dispensing unit in order to open the door. The hand sanitizer dispensing unit and additional instructions are preferably located to allow even a first-time user to quickly appreciate that activation of the dispensing unit and receipt of a hand sanitizer is required to allow passage through the door threshold.

### 2. Doors

Doors used in the system or methods of the present invention, when in a closed position and blocking access past a door threshold, can be either locked or unlocked. In an unlocked state, use of the door opening system of the invention causes a door opening device (as discussed below) to open the door upon receipt of a signal from the hand sanitizer dispensing unit. In this instance, the door opening device allows for the convenient passage of a user through a door threshold without the need to manually open the door. In addition, the system and methods of the invention can be applied to a locked door (i.e., a door further equipped with a door locking device, as discussed below). In this case, activation of the door opening system, by triggering the dispensing unit as described above, can unlock the door to allow the user to pass through the door manually (e.g., by pushing or pulling open a door). Alternatively, activation of the dispensing unit can both unlock the door and activate a door opening device to automatically open the door.

Elevator doors can also be used in the system of the invention. Typically, elevators must be “called” by pressing a button near the exterior door. When coupled to the system of the invention, activation of a hand sanitizer dispensing unit placed near an elevator door or bank can be used as an elevator call signal.

### 3. A Door Opening Device

A door opening device of the invention is a mechanical device that opens one or more doors upon receipt of a signal from a hand sanitizer dispensing unit. The device can use a motor, engine, or hydraulic arm to effect the movement of the door. Door opening devices (e.g., mechanical devices, such as hydraulic door openers) are known in the art (e.g., U.S. Pat. Nos. 2,591,693, 3,708,915, 4,660,324, and 4,348,835, herein incorporated by reference). Door opening devices, are colloquially known as “automatic door openers” and are frequently encountered when entering supermarkets and other retail spaces. Door opening devices can include a power supply (e.g., an electrical current) if necessary for operation. Door opening devices can include a manual override or fail-safe mechanism that allows the door or doors to be opened in the event of mechanical or electrical failure (e.g., loss of electrical power).

### 4. A Door Locking Device

Door locking devices (e.g., a door lock) of the invention can be based on a mechanical, electrical, or magnetic designs and serve to prevent the movement of a door when in a closed and locked position. Door locks and locking mechanisms suitable for inclusion in the system of the invention are known to skilled artisans that install and maintain doors. Door locking devices can include a power supply (e.g., an electrical current) if necessary for operation. Door locking devices can include a manual override or fail-safe mechanism that allows the door or doors to be opened in the event of mechanical or electrical failure (e.g., loss of electrical power).

### 5. A Signal Connection Between the Dispenser Unit and Door Opening or Locking Device

Upon activation, the hand sanitizer dispensing unit of the invention transmits a signal, through a signal connection, to the door opening or locking device, as defined herein. The signal connection can be wired (e.g., copper electrical wires that physically connect the dispensing unit to the door opening or locking device) to allow the transmission of an electrical signal. Alternatively, the signal connection can be wireless, such as a radio or infrared signal. In this case, the dispensing unit contains a wireless transmitter that emits a signal upon activation of the unit. Upon receipt of a signal through the signal connection, the door opening or locking device is activated, allowing passage of the user through the door threshold.

### Advantages

The door opening system of the invention compels a person at person at risk of transmitting or becoming infected with a microbial pathogen to apply an effective amount of hand sanitizer to their hands before passing through a doorway or door threshold. Traditionally, in healthcare (e.g., hospitals), food preparation and service (e.g., kitchens), and public transportation (e.g., cruise ships, airports) settings, hand-washing is encouraged to limit the transmission of microbial infections. Stand alone hand sanitizer dispensing units are also frequently placed in healthcare settings to reduce the spread of disease-causing microbes. In the absence of compulsion (e.g., employee surveillance or hand-washing “checklists”), many individuals do not properly disinfect their hands, either by hand-washing or by using a hand sanitizer, frequently enough to reduce the transmission of infectious microbes. The system of the present invention provides a convenient but compulsory method to reduce or eliminate the transmission of infectious microbes amongst humans. The application of the system of the invention to several settings is considered below:

### Healthcare Facilities

The system of the invention is ideally used in locations where it is extremely important to control the spread of microbes and pathogens that cause healthcare-associated infections. As discussed, hospitals and other healthcare facilities (e.g., nursing homes, outpatient clinics, and blood-donation centers) suffer from high transmission rates of microbial infections, greatly increasing the morbidity and mortality of those receiving medical treatment at these facilities. Doors that separate patient areas (e.g., patient rooms, surgical procedure rooms, and intensive care units) from common areas, such as hallways or waiting rooms can be equipped with the system of the invention. The system of the invention is particularly useful used to limit access to immunocompromised medical patients (e.g., patients that have received ablative irradiation prior to transplantation or patients suffering from acquired immunodeficiency syndrome (AIDS)).

### Food Preparation

It is recognized that transmission of microbial pathogens from the hands to food products is a frequent means of communicating diseases, often responsible for severe illness or death. Numerous episodes involving fatalities due to contamination of food products during food handling have increased the public awareness of the serious consequences of poor hygiene in food preparation, distribution, or consumption settings. Although proper hand-washing hygiene is typically stressed, the reality is often poor compliance by the food preparers, handlers, cooks, and servers. Food preparation and service facilities such as slaughterhouses; food (e.g., meat, poultry, vegetable, fruit, or dairy products intended for human or other animal consumption) collection, processing, packing, and storage facilities; kitchens (e.g., a home, school, hospital, or cafeteria kitchen); cafeterias; and restaurants are particularly vulnerable to harboring and transmitting microbial infections. In these settings, the use of the door opening system of the invention would compel food preparation and service personnel (e.g., butchers, harvesters, meat packers, cooks, and waiters) to apply a hand sanitizer to their hands prior to entering or exiting a food preparation area.

### Transportation

Modes of transportation, especially public transportation vehicles and transportation stations (e.g., an airport), are frequent locations for the spread of infection microbes. The close proximity of many people, often in the absence of hand-washing facilities, makes these environments ideal for the transmission of disease-causing pathogens. Holiday cruise ships have been demonstrated to be especially vulnerable by the frequent and intense outbreaks of gastrointestinal infections, especially noroviral infections. The use of the system of the invention throughout a cruise ship or airplane can reduce or eliminate the transmission of infectious microbes. For example, restroom doors could be equipped with the system of the invention to compel anyone exiting a restroom to apply a hand sanitizer.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications mentioned in this specification are herein incorporated by reference to the same extent as if each independent publication or patent application was specifically and individually indicated to be incorporated by reference in their entirety.

What is claimed is:

1. A door opening system comprising:
a) a hand sanitizer dispensing unit that releases a hand sanitizer upon activation;
b) a door with a door opening or locking device; and
c) an electrical, mechanical, or wireless signal connection between said hand sanitizer dispensing unit and said opening or locking device;
wherein said door opening or locking device is adapted to be activated to allow movement of said door upon activation of said hand sanitizer dispensing unit.

2. The system of claim 1, wherein said hand sanitizer comprises a chemical or radiological sanitizer.

3. The system of claim 2, wherein said chemical sanitizer is selected from the group consisting of triclosan, ethanol, isopropanol, n-propanol, povidone-iodide, chlorhexidine gluconate, octenidine, chloroxylenol, hexachlorophene, alkyl benzalkonium chloride, benzethonium chloride, cetrimide, and cetylpyridium chloride.

4. The system of claim 2, wherein said chemical sanitizer is ethanol.

5. The system of claim 2, wherein said chemical sanitizer is triclosan.

6. The system of claim 2, wherein said chemical hand sanitizer is applied with a towel, towelette, or hand wipe.

7. The system of claim 2, wherein said radiological sanitizer is ultraviolet light.

8. The system of claim 1, wherein said door is an elevator door.

9. The system of claim 1, wherein said wireless signal connection comprises a radio or infrared connection.

10. The system of claim 1, wherein said door opening or locking device further comprises a manual override mechanism, wherein use of said mechanism allows movement of the door independent of said activation of said hand sanitizer dispensing unit.

11. The system of claim 1, wherein said hand sanitizer dispensing unit is mounted on said door.

12. The system of claim 1 further comprising instructions for the use of said system.

13. The system of claim 1, wherein said door is contained in a hospital, medical facility, nursing home, school, daycare center, library, museum, public meeting or working space, retail store, grocery store, restaurant, cafeteria, kitchen, food production or processing facility, public transportation station, or public transit vehicle.

14. The system of claim 1, wherein said door is contained in a hospital.

15. The system of claim 1, wherein said door is contained in a hospital emergency room (ER), intensive care unit (ICU), operating room (OR), surgical preparation room, outpatient facility, birthing room, pharmacy, cafeteria, restroom, elevator bank, or waiting room.

16. The system of claim 1, wherein said door is contained in an airport, train station, subway station, or bus station.

17. The system of claim 1, wherein said door is contained in an airplane, a train, a subway train, a bus, or boat.

18. The system of claim 17, wherein said boat is a cruise ship.

19. The system of claim 1, wherein said door separates medical caregivers from medical patients.

20. The system of claim 19, wherein said caregivers comprise physicians, nurses, medical technicians, pharmacists, or medical students.

21. A method of reducing microbial infection comprising activation of a hand sanitizer dispensing unit mounted on or proximal to a door with a door opening or locking device, wherein said activation allows movement of said door.
